(19) United States
(12) Patent Application Publication
(10) Pub. No.: US 2017/0191028 A1
(43) Pub. Date: Jul. 6, 2017

(54) METHODS OF DEVELOPING PLANT SOMATIC EMBRYOS

(71) Applicant: Weyerhaeuser NR Company, Seattle, WA (US)

(72) Inventor: Pramod K. GUPTA, Federal Way, WA (US)

(73) Assignee: Weyerhaeuser NR Company, Seattle, WA (US)

(21) Appl. No.: 15/347,628

(22) Filed: Nov. 9, 2016

Related U.S. Application Data
(60) Provisional application No. 62/273,837, filed on Dec. 31, 2015.

(51) Int. Cl. C12N 5/04

(52) U.S. Cl. CPC C12N 5/04; C12N 2523/00

(57) ABSTRACT

Methods of developing plant somatic embryos include incubating the plant somatic embryos at a first temperature for a first period of time followed by storing the plant somatic embryos at a second temperature for a second period of time. The first temperature can be in the range of about 23° C. to 30° C. and leads to rapid plant somatic embryo growth. The second temperature can be in the range of about 8° C. to 15° C. and slows plant somatic embryo development. Plant somatic embryos developed in this manner and implanted in artificial seeds show germination rates similar to zygotic embryos implanted in artificial seeds.

### CROSS REFERENCE AND RELATED APPLICATION

This application is a nonprovisional claiming priority to U.S. Provisional Patent Application No. 62/273,837, filed Dec. 31, 2015, which is incorporated herein by reference in its entirety. To the extent the foregoing application and/or any other materials incorporated herein by reference conflict with the present disclosure, the present disclosure controls.

### TECHNICAL FIELD

The present technology is related to methods of developing plant somatic embryos. In particular, the present technology is related to developing plant somatic embryos at different temperatures during different stages of somatic embryo development to improve somatic embryo quality.

### BACKGROUND

The demand for coniferous trees, such as pines and firs, to make wood products continues to increase. One proposed solution to this problem is to identify individual trees that possess desirable characteristics, such as rapid rate of growth, and produce numerous, genetically identical, clones of the trees by somatic cloning.

Somatic cloning is the process of creating genetically identical trees from tree tissue other than male and female gametes. In one approach to somatic cloning, plant tissue is cultured in an initiation medium that incudes hormones, such as auxins and cytokinins. These hormones initiate formation of embryogenic cells that subsequently develop into somatic embryos. The somatic embryos are then developed into mature embryos that can be, for example, placed within artificial seeds and sown into the soil where they germinate to produce seedlings.

Methods of assisting somatic embryo development typically include developing somatic embryos at around room temperature (20° C.) for a period of time during which the somatic embryos grow in size and mature. Regardless of such efforts to improve somatic embryo development, somatic embryos do not germinate at the same rate as zygotic embryos implanted in artificial seeds. Accordingly, methods of developing somatic embryos that improve the quality of somatic embryos and bring their rate of germination closer to that of zygotic embryos implanted in artificial seeds are desired.

### BRIEF DESCRIPTION OF THE DRAWINGS

FIG. 1 is a flow chart illustrating a method of developing plant somatic embryos according to various embodiments described herein;

FIGS. 2-4 are graphs illustrating experimental data from an experiment carried out in accordance with various embodiments of the method described herein; and

FIGS. 5-10 are photographs of plant somatic embryos at various stages of the experiment referred to above with respect to FIGS. 2-9.

### DETAILED DESCRIPTION

Several embodiments of a method of developing plant somatic embryos are described below. In some embodiments, the method includes developing plant somatic embryos at a first temperature for a period of time, followed by developing the plant somatic embryos at a second temperature less than the first temperature for a second period of time. For example, the method includes developing plant somatic embryos at the first temperature until the embryos reach the cotyledon stage, and then developing the plant embryos at the lower second temperature. The first temperature can be in range of from about 23° C. to about 30° C., such as about 25° C., and the second temperature can be in the range of from about 8° C. to about 15° C., such as about 12° C. In some embodiments, the plant somatic embryos are developed at 25° C. until the somatic embryos enter the cotyledon stage, after which point they are developed at 12° C. The plant somatic embryos undergo rapid growth at the higher temperature range, while the development of the somatic embryo slows at the lower temperature range.

The present technology improves the development of plant somatic embryos by mimicking zygotic embryo growth, which typically involves rapid development until the cotyledon stage followed by very slow growth during late development and maturation of the zygotic embryo. As a result of the methods described in the present application, larger embryos are produced during the initial storing step as compared to when room temperature is used during early somatic embryo development. These larger embryos subsequently have a higher quality and germinate when implanted in artificial seeds at a rate similar to zygotic embryos implanted in artificial seeds.

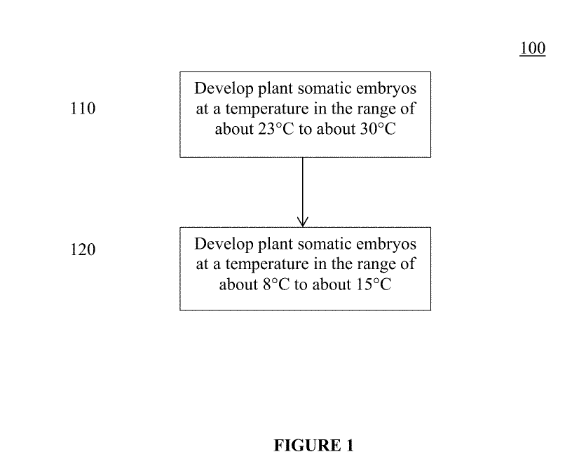

With reference to FIG. 1, a method 100 of developing plant somatic embryos according to several embodiments described below includes developing plant somatic embryos at a first temperature (block 110), such within the range of from 23° C. to 30° C., followed by developing the plant somatic embryos at a second temperature less than the first temperature (block 120), such as within the range of 8° C. to 15° C.

The plant somatic embryos can generally include any type of plant somatic embryo that has not yet begun or is in the early stages of somatic embryo development before being developed at the first temperature (block 110). The plant somatic embryo can be for any suitable type of plant. Examples of plant somatic embryos suitable for use in the methods described in the present application include, but are not limited to, loblolly pine somatic embryos, gymnosperm somatic embryos, conifer somatic embryos, and Douglas-fir somatic embryos.

Table 1 below provides example compositions of various media that can be used for the development of plant somatic embryos according to methods described herein. Prior to being developed at the first temperature (block 110), the plant somatic embryos can undergo various initial development stages which generally include providing the plant somatic embryos with different types of media. For example, the plant somatic embryos can be sequentially provided with an initiation media (for example, such as described in U.S. Published Application No. 2014/0178999), a solid maintenance and multiplication media (BM2), and a liquid maintenance and multiplication media (BM3), followed by being subjected to a rinsing stage (BM4). Following the rinsing stage, a development media (BM5) is used for the first temperature development stage and the second temperature development stage. Subsequently, a cold stratification stage (BM6), and the germination stage (BM7) can be carried out. The compositions shown in the Table 1 are examples of suitable media used for various stages of plant somatic embryo development, and other suitable media compositions can be used for the various stages.

TABLE 1
Loblolly Pine Media Comparison

| | BM 2 | BM 3 | | | | BM 7 |
|---|---|---|---|---|---|---|
| BM 1 | “3×” | Liquid | BM 4 | BM 5 | BM 6 | Germ |
| 1× Maint. | Maint. | Maint. | Rinse | Dev. | Cold | 185 |

Salts (mg/L)

| | | | | | | | |
|---|---|---|---|---|---|---|---|
| NH4NO3 | 150 | 150 | 150 | 150 | 150 | 150 | 206.25 |
| KNO3 | 909.9 | 909.9 | 909.9 | 909.9 | 909.9 | 909.9 | 1170 |
| Ca(NO3)2•4H2O | 236.15 | 236.15 | 236.15 | 236.15 | 236.15 | 236.15 | |
| MgSO4•7H2O | 246.5 | 246.5 | 246.5 | 246.5 | 246.5 | 246.5 | 185 |
| Mg(NO3)2•6H2O | 256.5 | 256.5 | 256.5 | 256.5 | 256.5 | 256.5 | |
| MgCl2•6H2O | 50 | 50 | 50 | 50 | 50 | 50 | |
| KH2PO4 | 136 | 136 | 136 | 136 | 136 | 136 | 85 |
| CaCl2•2H2O | 50 | 50 | 50 | 50 | 50 | 50 | 220 |
| KI | 4.15 | 4.15 | 4.15 | 4.15 | 4.15 | 4.15 | 0.415 |
| H3BO3 | 15.5 | 15.5 | 15.5 | 15.5 | 15.5 | 15.5 | 3.1 |
| MnSO4•H2O | 10.5 | 10.5 | 10.5 | 10.5 | 10.5 | 10.5 | 8.45 |
| ZnSO4•7H2O | 14.4 | 14.4 | 14.4 | 14.4 | 14.4 | 14.4 | 4.3 |
| Na2MoO4•2H2O | 0.125 | 0.125 | 0.125 | 0.125 | 0.125 | 0.125 | 0.125 |
| CuSO4•5H2O | 0.125 | 0.125 | 0.125 | 0.125 | 0.125 | 0.125 | 0.0125 |
| CoCl2•6H2O | 0.125 | 0.125 | 0.125 | 0.125 | 0.125 | 0.125 | 0.0125 |
| FeSO4•7H2O | 27.87 | 27.87 | 27.87 | 27.87 | 27.87 | 27.87 | 13.93 |
| Na2EDTA | 37.26 | 37.26 | 37.26 | 37.26 | 37.26 | 37.26 | 18.63 |

Vitamins/Amino Acids (mg/L)

| | | | | | | | |
|---|---|---|---|---|---|---|---|
| Nicotinic Acid | 0.5 | 0.5 | 0.5 | 0.5 | 0.5 | 0.5 | 0.5 |
| Pyridoxine HCl | 0.5 | 0.5 | 0.5 | 0.5 | 0.5 | 0.5 | 0.5 |
| Thiamine HCl | 1 | 1 | 1 | 1 | 1 | 1 | 1 |
| Glycine | 2 | 2 | 2 | 2 | 2 | 2 | 2 |
| L-proline | | | | 100 | 100 | 100 | |
| L-asparagine | | | | 100 | 100 | 100 | |
| L-arginine | | | | 50 | 50 | 50 | |
| L-alanine | | | | 20 | 20 | 20 | |
| L-serine | | | | 20 | 20 | 20 | |
| PEG 8000 mw | | | | | 100000 | | |

Sugar/Agar mg/L

| | | | | | | | |
|---|---|---|---|---|---|---|---|
| Myo-Inositol | 200 | 200 | 200 | 100 | 100 | 100 | 100 |
| Casein hydrolysate | 500 | 500 | 500 | 500 | 500 | 500 | |
| L-glutamine | 1000 | 1000 | 1000 | 1000 | 1000 | 1000 | |
| Sucrose | | | | | | 25000 | 20000 |
| Maltose | 30000 | 30000 | 30000 | 25000 | 25000 | | |
| Glucose | | | | | 10000 | | |
| Gelzan | 1600 | 1600 | | | 2500 | 2500 | |
| TC Agar | | | | | | | 8000 |
| Activated carbon | | | | | 1000 | 1000 | 2500 |

Hormones mg/L

| | | | | | | | |
|---|---|---|---|---|---|---|---|
| ABA | | | | | 25 | | |
| NAA | | | | | | | |
| 2,4-D | 1.1 | 3.0 | 1.1 | | | | |
| BAP | 0.1 | 0.25 | 0.1 | | | | |
| Kinetin | 0.1 | 0.25 | 0.1 | | | | |
| pH | 5.7 | 5.7 | 5.7 | 5.7 | 5.7 | 5.7 | 5.7 |

Once media such as BM2 to BM4 are used, the somatic embryos can be plated on a development plate that includes development media, such as, but not limited to, BM5. Any method of plating the somatic embryos can be used and any suitable equipment can be used to carry out the plating. In some embodiments, the plating method and plating equipment are designed to provide an even distribution of embryos across the development plate and suitable spacing between adjacent embryos.

The development media used on the development plate is generally not limited, and can include any media suitable for growing plant somatic embryos. One example of a suitable development media is BM5 described in Table 1 above. Once the plant somatic embryos are plated and provided with the development media, the development plates can be stored in storage containers, such as Cambro® boxes. In some embodiments, a single development plate is housed in a storage container.

In one embodiment, the plant somatic embryos are developed at a first temperature of about 23° C. to 30° C., and more specifically about 25° C., for a first period of time. The manner of developing the plant somatic embryos within this range is generally not limited. In some embodiments, development of the embryos is carried out by incubating the plant somatic embryo in an enclosed environment that is maintained at a temperature within the range of from 23° C. to 30° C., such as 25° C., for a first period of time. The enclosed environment can be a room where the entirety of the room is maintained at the desired temperature, or can be a free-standing vessel maintained at the desired temperature and in which the plant somatic embryos can be incubated. Any manner of maintaining the temperature at the desired temperature can be used provided the temperature is held at a constant temperature or within a specific temperature range during the time the somatic embryos are incubated within the enclosed environment. In some embodiments, the enclosed environment is kept dark in order to prevent premature germination of the embryos.

In some embodiments, the first period of time is based on the amount of time that allows plant somatic embryos to grow in size. In some embodiments, the plant somatic embryos are developed at a temperature within the range of 23° C. to 30° C. until one or more of the plant somatic embryos enter the cotyledon stage of embryo development. Plant somatic embryos of fir and/or pine trees stored at, e.g., 25° C. generally take from 3 weeks to 6 weeks to enter the cotyledon stage of development, though shorter or longer times are also possible.

Any manner of determining when the plant somatic embryos enter the cotyledon stage can be used, including visual inspection of the plant somatic embryos. For example, visual inspection for embryos with an opaque color can be used as an indicator that the plant somatic embryos have entered the cotyledon development stage. In some embodiments where multiple plant somatic embryos are plated on a development frame, the plant somatic embryos are developed at a temperature in the range of from 23° C. to 30° C. until a majority of the plant somatic embryos on the development plate have entered the cotyledon stage.

In one embodiment of the lower temperature step (block 120), the plant somatic embryos previously developed at, e.g., 25° C. are subsequently developed at a temperature within the range of from 8° C. to 15° C., such as 12° C., for a period of time.

The manner of developing the plant somatic embryos within a temperature of from 8° C. to 15° C. can generally be similar or identical to the manner of developing the plant somatic embryos at a temperature within the range of from 23° C. to 30° C. as described previously, with the exception of the temperature being maintained within the lower temperature range. Thus, in some embodiments, the plant somatic embryos are developed by incubating the embryos in an enclosed room or free-standing vessel that is capable of maintaining a temperature within the range 8° C. to 15° C.

In some embodiments, transitioning from the higher temperature process to the lower temperature process includes transporting the plant somatic embryos from a first enclosed environment maintained at the first temperature (e.g., 25° C.) to a second enclosed environment maintained at the second temperature (e.g., 12° C.). The transitioning of the plant somatic embryos from the high temperature process (block 110) to the low temperature process (block 120) should generally be carried out as quickly as possible so that the plant somatic embryos are not subjected to a third temperature for any significant period of time. Alternatively, an enclosed environment can be provided that is capable of quickly altering the temperature from within the higher temperature range to within the lower temperature range so that the plant somatic embryos do not need to be transported to a different location as part of transitioning between the high and low temperature processes.

The plant somatic embryos can be developed at a temperature in the range of from 8° C. to 15° C. for a period of time during which development of the plant somatic embryos is slowed after the plant somatic embryos have entered the cotyledon stage.

In one embodiment, the plant somatic embryos are developed at a temperature within the range of from 8° C. to 15° C. for whatever period of time is necessary such that high and low temperature processes are carried out in exactly 12 weeks. For example, if the high temperature process (block 110) is carried out in 5 weeks (such as if it takes 5 weeks for the plant somatic embryos to enter the cotyledon stage), then the low temperature process (block 120) is carried out in 7 weeks, regardless of whether germination has occurred in any of the plant somatic embryos at the end of the 7 weeks.

Compared to plant embryos developed at room temperature, plant somatic embryos developed according to methods described above tend to have better quality and germinate when implanted in artificial seeds at a rate similar to zygotic embryos implanted in artificial seeds. In some experiments, the rate of germination for plant somatic embryos developed according to the methods described above improved 80% over plant somatic embryos developed at room temperature.

### Example

A method of developing plant somatic embryos was carried out on two different somatic embryo clones (clone A and clone B). Each clone was plated on 15 development plates, and each plate was then housed in a Cambro® box. Three of the 15 boxes per clone were then randomly selected for development.

Three treatment protocols were developed as summarized in Table 2. In treatment 1 (Control), the Cambro® box having the plated somatic embryos housed therein was incubated in a room at room temperature (20° C.) until a majority of the plated plant somatic embryos were observed to have entered the cotyledon stage, at which point the Cambro® box was incubated in a room at 12° C. In treatment 2, the Cambro® box having the plated somatic embryos housed therein was incubated in a room at 25° C., and was transferred to a room at 12° C. at the same time as Treatment 1 was transferred, regardless of whether the somatic embryos had entered the cotyledon stage. In treatment 3, the Cambro® box having the plated somatic embryos housed therein was incubated in a room at 25° C. until a majority of the plated plant somatic embryos were observed to have entered the cotyledon stage, at which point the Cambro® box was incubated in a room at 12° C. For all treatments, the Cambro® boxes were removed from the 12° C. room after a total of 12 weeks had passed from the time the Cambro® box was first incubated in the 20° C. or 25° C. room. Visual inspection was used to determine when a majority of the somatic embryos had entered the cotyledon stage.

TABLE 2

| Treatment | Initial Dev Temp | Timing to cool development |
|---|---|---|
| 1 | 20° C. - Room | majority of the population |
| (Control) | Temperature | to be at cotyledon stage |
| 2 | 25° C. | Same time as Treatment 1 |
| 3 | 25° C. | majority of the population |
| | | to be at cotyledon stage |

Table 3 summarizes the time each box was stored in the 20° C. or 25° C. room and the time each box was stored in the 12° C. room for all three treatments of each genotype tested.

TABLE 3

| | | | Length of time | Length of time | Total |
|---|---|---|---|---|---|
| | | | in dev temp | in 12 degree | dev. time |
| | Genotype | Treatment | (wks) | (wks) | (wks) |
| | Clone A | 1 | 4.86 | 7.14 | 12 |
| | | 2 | 4.86 | 7.14 | 12 |
| | | 3 | 4 | 8 | 12 |
| 13 | 1263/ | 1 | 5.71 | 6.29 | 12 |
| | 529.4 | 2 | 5.71 | 6.29 | 12 |
| | | 3 | 3.86 | 8.14 | 12 |

The responses considered for the analysis of this experiment were category 1 germination fraction, number of category 1 germinants/mL, and yield/development frame, Category 1 germination fraction was analyzed with a generalized mixed linear model. Germinants/mL and yield/development frame were analyzed with a linear mixed model after taking the log transformation. Treatment means and confidence intervals were transformed back to the natural scale for all responses

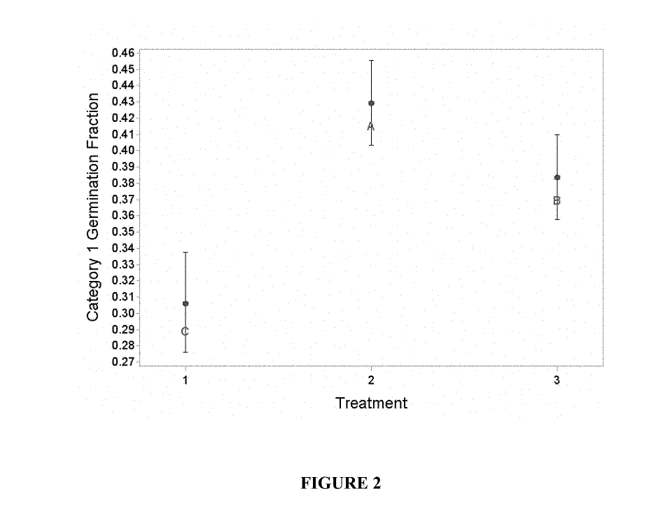

FIG. 2 shows the means and confidence levels for category 1 germination fraction by treatment and genotype. Error bars are 90% confidence limits. There is strong evidence of a treatment effect (p-value=0.0005). There is strong evidence of a genotype effect (p-value=<0.0001). Treatment LSmeans and pairwise comparisons are given in Table 4. L90 and U90 are the lower and upper 90% confidence limits, respectively, for each mean. The column “Test at α=0.10” summarizes test results comparing combined means. Means with the same symbol are not statistically different at α=0.10.

TABLE 4

| | | Test at | | |
|---|---|---|---|---|
| Treatment | Mean | α = 0.10 | L90 | U90 |
| 1 | 0.306 | C | 0.276 | 0.338 |
| 2 | 0.429 | A | 0.403 | 0.456 |
| 3 | 0.383 | B | 0.358 | 0.410 |

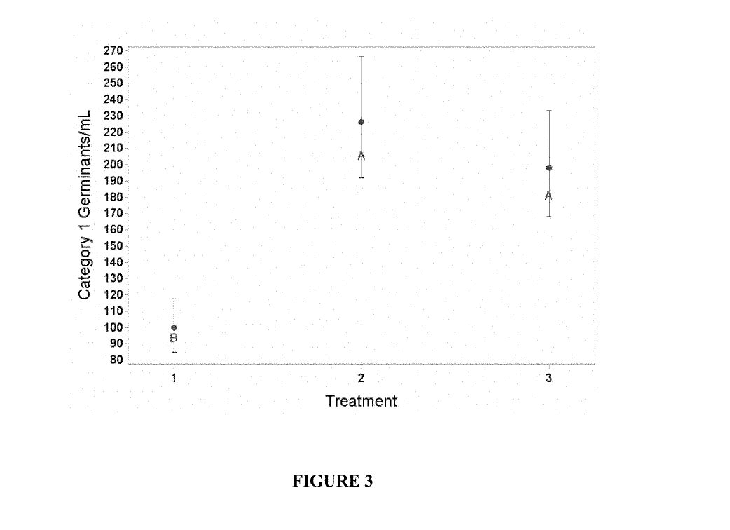

FIG. 3 shows the means and confidence levels for number of category 1 germinants/mL by treatment and genotype. Error bars are 90% confidence limits. There is strong evidence of a treatment effect (p-value <0.0001). There is moderate evidence of a genotype-batch effect (p-value=0.04). Treatment LSmeans and pairwise comparisons are given in Table 5. L90 and U90 are the lower and upper 90% confidence limits, respectively, for each mean. The column “Test at α=0.10” summarizes test results comparing combined means. Means with the same symbol are not statistically different at α=0.10.

TABLE 5

| | | Test at | | |
|---|---|---|---|---|
| Treatment | Mean | α = 0.10 | L90 | U90 |
| 1 | 99.9 | B | 84.8 | 117.6 |
| 2 | 226.2 | A | 192.1 | 266.2 |
| 3 | 198.1 | A | 168.3 | 233.2 |

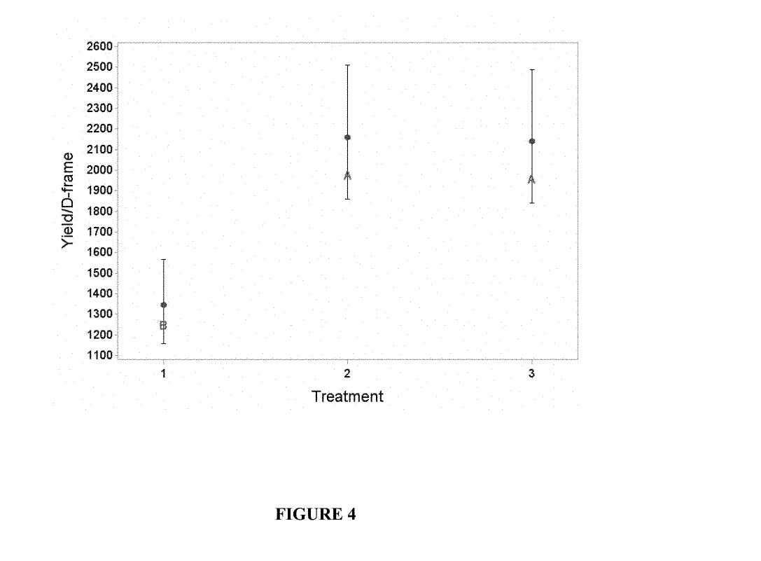

FIG. 4 shows the means and confidence levels for yield/development frame by treatment and genotype. Error bars are 90% confidence limits. There is strong evidence of a treatment effect (p-value=0.002). There is suggestive evidence of a genotype-batch effect (p-value=0.13). Treatment LSmeans and pairwise comparisons are given in Table 6. L90 and U90 are the lower and upper 90% confidence limits, respectively, for each mean. The column “Test at α=0.10” summarizes test results comparing combined means. Means with the same symbol are not statistically different at α=0.10.

TABLE 6

| | | Test at | | |
|---|---|---|---|---|
| Treatment | Mean | α = 0.10 | L90 | U90 |
| 1 | 1347.0 | B | 1158.7 | 1565.9 |
| 2 | 2160.7 | A | 1858.6 | 2511.8 |
| 3 | 2139.8 | A | 1840.7 | 2487.6 |

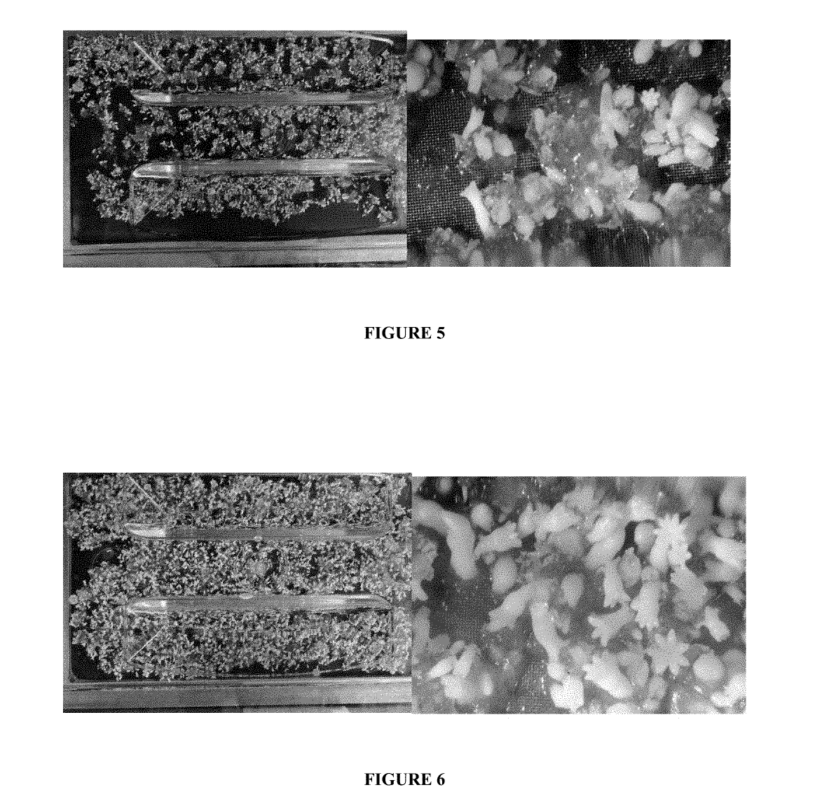

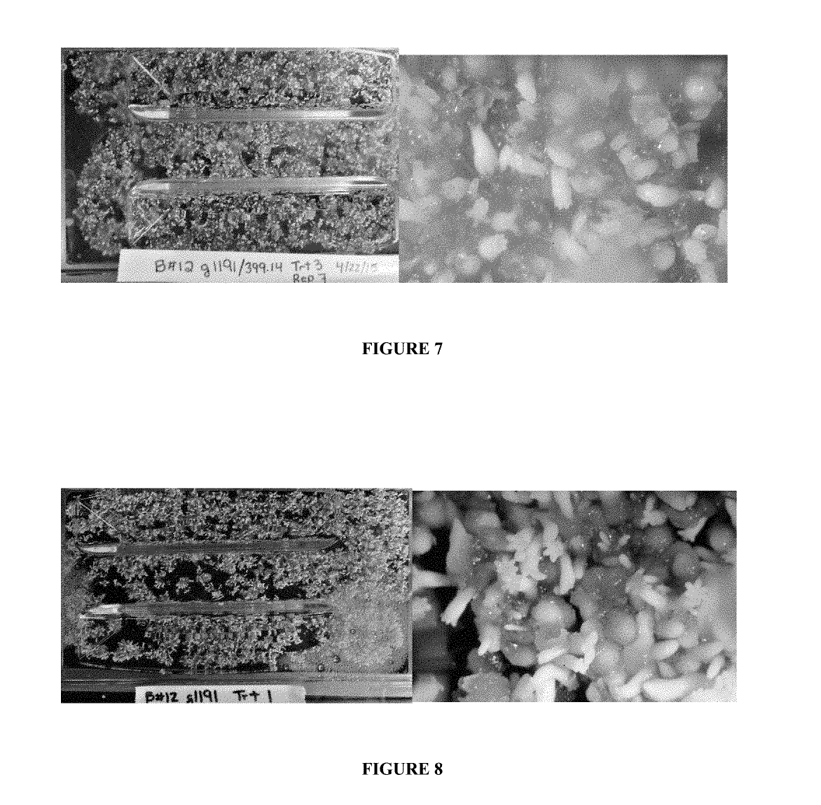

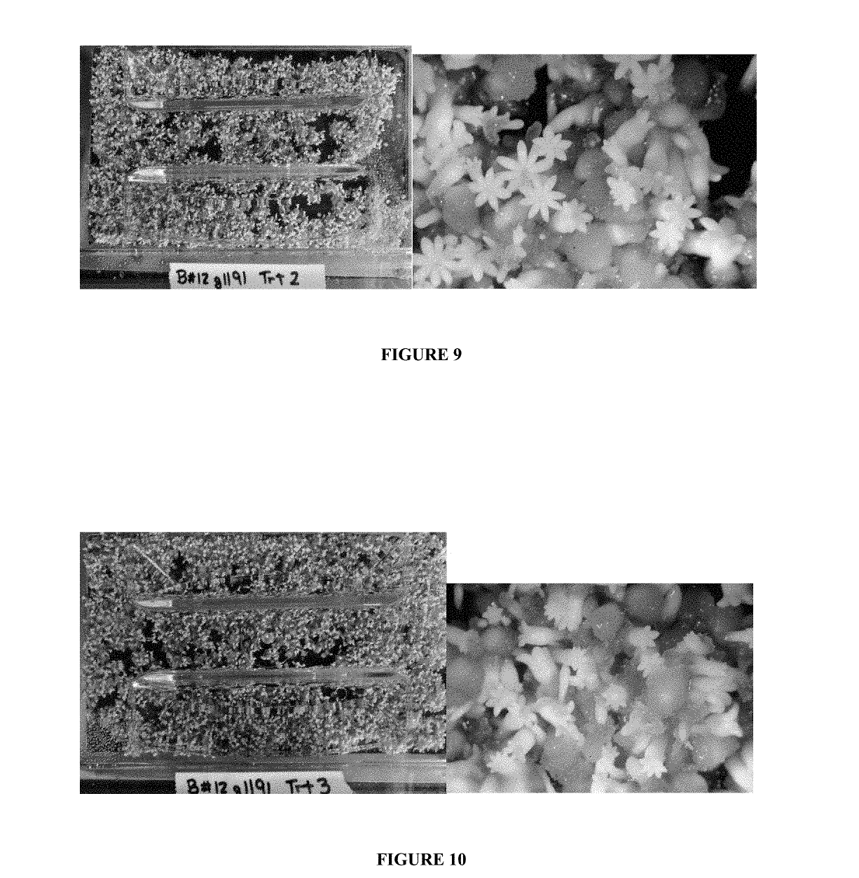

FIGS. 5-10 are photographs of the embryos are various different stages of the experiment described above.

FIG. 10 shows Clone A, treatment 1, at 4.86 weeks; FIG. 11 shows Clone A, treatment 2, at 4.86 weeks; and FIG. 12 shows Clone A, treatment 3, at 4 weeks. These photographs thus show the embryos at the end of being stored at 20° C. or 25° C.

FIG. 13 shows Clone A, treatment 1, at 12 weeks; FIG. 14 shows Clone A, treatment 2, at 12 weeks; and FIG. 15 shows Clone A, treatment 3, at 12 weeks. These photographs thus show the embryos at the end of being stored at 12° C.

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A method of developing a plant somatic embryo, comprising:
(a) developing the plant somatic embryo at a temperature in the range of about 23° C. to about 30° C.; and
(b) developing the plant somatic embryo at a temperature in the range of about 8° C. to 15° C.

2. The method of claim 1, wherein developing the plant somatic embryo at a temperature in the range of about 23° C. to about 30° C. comprises incubating the plant somatic embryo at a temperature in the range of about 23° C. to about 30° C. until the plant somatic embryo enters the cotyledon stage.

3. The method of claim 1, wherein developing the plant somatic embryo at a temperature in the range of about 23° C. to about 30° C. comprises incubating the plant somatic embryo at a temperature in the range of about 23° C. to about 30° C. in a first environment and developing the plant somatic embryo at a temperature in the range of about 8° C. to 15° C. comprises incubating the plant somatic embryo at a temperature in the range of about 8° C. to 15° C. in a second environment, and the method further comprises transferring the plant somatic embryo from the first environment to the second environment.

4. The method of claim 1, wherein the method further comprises:
plating the plant somatic embryo on a development plate having development media disposed thereon prior to developing the plant somatic embryo at a temperature in the range of about 23° C. to about 30° C.

5. The method of claim 1, wherein developing the plant somatic embryo at a temperature in the range of about 23° C. to about 30° C. comprises developing the plant somatic embryo at a temperature of about 25° C.

6. The method of claim 5, wherein developing the plant somatic embryo at a temperature of about 25° C. comprises developing the plant somatic embryo at a temperature of about 25° C. for a period of from 3 to 6 weeks.

7. The method of claim 1, wherein developing the plant somatic embryo at a temperature in the range of about 8° C. to about 15° C. comprises developing the plant somatic embryo at a temperature of about 12° C.

8. A method of developing plant somatic embryos, comprising:
(a) developing the plant somatic embryos at a temperature of about 25° C. until a majority of the plant somatic embryos enter the cotyledon stage; and
(b) developing the plant somatic embryos at a temperature of about 12° C.

9. The method of claim 8, wherein developing the plant somatic embryos at a temperature of about 25° C. comprises incubating the plant somatic embryos at a temperature of about 25° C. in a first environment and developing the plant somatic embryos at a temperature of about 12° C. comprises incubating the plant somatic embryos at a temperature of about 12° C. in a second environment, and the method further comprises transferring the plant somatic embryos from the first environment to the second environment.

10. The method of claim 8, wherein the method further comprises:
plating the plant somatic embryos on a development plate having development media disposed thereon prior to developing the plant somatic embryos at a temperature of about 25° C.
